(19) United States
(12) Patent Application Publication
(10) Pub. No.: US 2011/0319780 A1
(43) Pub. Date: Dec. 29, 2011

(54) Method for identifying a characteristic profile of an R-wave in an EKG signal and a computer program product as well as an electronically readable data medium for performing the method

(76) Inventors: Michael Frank, Erlangen (DE); Jürgen Rössler, Erlangen (DE)

(21) Appl. No.: 13/164,968

(22) Filed: Jun. 21, 2011

(30) Foreign Application Priority Data
Jun. 24, 2010 (DE) 10 2010 024 965.3

(51) Int. Cl. A61B 5/0456

(52) U.S. Cl. 600/521

(57) ABSTRACT

A method for identifying a characteristic profile of an R-wave in an EKG signal is proposed. A temporal sequence of measurement values is recorded and stored with associated time value. A number of values for identified temporal derivative and their respective time values is identified in the stored measurement values. One of the values for identified temporal derivative is selected as an exemplary value. The selection includes at least one plausibility test. A sub-sequence of the stored measurement values is selected as a characteristic profile as a function of the time value associated with the exemplary value. The combination of identifying possible values by their derivative with a plausibility test makes the method particularly robust.

### CROSS REFERENCE TO RELATED APPLICATIONS

This application claims priority of German application No. 10 2010 024 965.3 filed Jun. 24, 2010, which is incorporated by reference herein in its entirety.

### FIELD OF THE INVENTION

The invention relates to a method for identifying a characteristic profile of an R-wave in an EKG signal, as well as a computer program product and an electronically readable data medium for performing the method.

### BACKGROUND OF THE INVENTION

EKG measurement apparatuses are used primarily for measuring and monitoring a patient's cardiac function, for which purpose the summation voltage of the electrical activity of the myocardial fibers is typically measured across at least two electrodes as what is termed an “EKG signal”. An ideal profile of such an EKG signal is shown by way of example in FIG. 1 as voltage U over time. According to Einthoven, characteristic profiles of the EKG signal are designated by the letters P, Q, R, S and T and generally reflect the different phases of a heartbeat.

There are other applications in addition to the pure monitoring of a patient's cardiac function. For example, EKG signals are also used in medical imaging applications for the purpose of generating trigger signals. During imaging, information about the cardiac phase is acquired via the EKG signal in order thereby to synchronize imaging with the cardiac activity. In particular with imaging methods that require a relatively long recording time, high-quality images of the heart or images of regions that are moved by the heartbeat can be recorded in this way.

EKG measurement apparatuses are also used for in-situ recording of EKG signals during an examination of a patient by means of a magnetic resonance device. In this case, however, operation in the magnetic resonance device imposes special requirements on the EKG measurement apparatus due to the strong gradient fields and high-frequency fields used there for imaging in order to prevent mutual interference between magnetic resonance device and EKG measurement apparatus. EKG measurement apparatuses that are magnetic-resonance-compatible in the aforementioned sense are available on the market.

Identifying R-waves in EKG signals is essential for reliable triggering. Such identification is, however, made more difficult e.g. as a result of T-wave overshoots occurring in the magnetic field.

Magnetic fields that change over time, as used in the magnetic resonance device as magnetic gradient fields for position encoding, also continue to represent a further major problem for reliable EKG signal measurement. According to the law of induction, such temporally fluctuating magnetic fields generate interference voltages which are coupled into the EKG signal recorded by the EKG electrodes as interference. Magnetically generated interference signals of this kind become superimposed on the EKG signal generated by the heart and distort said signal.

Such interference is extremely undesirable. Reliable detection of the R-wave of the EKG signal is necessary in order to synchronize a recording of a magnetic resonance image with the heartbeat. The interference signals can be erroneously interpreted as an R-wave, for example due to their often similar shape, and consequently can incorrectly initiate a triggering of a recording of a magnetic resonance image. On the other hand it can also happen that a “real” R-wave is not detected as such due to the superimposed noise signals. This frequently leads to a significant deterioration in image quality.

Prior art attempts to solve this problem consisted in subjecting signals interpreted as a possible R-wave to a simple threshold value check in addition prior to a triggering. This threshold value check generally provides a maximum value that is not to be exceeded and a minimum value that is not to be undershot. If the maximum value is exceeded, it is assumed that interference has been coupled in due to the gradient fields. If the minimum value is undershot, it is assumed that a T-wave has erroneously been interpreted as an R-wave. In both cases no trigger signal is output.

### SUMMARY OF THE INVENTION

The object of the invention is therefore to specify a method, a computer program product and an electronically readable data medium, which permit reliable detection of R-waves of EKG signals measured in a magnetic resonance device.

According to the invention the object is achieved by a method, a computer program product and an electronically readable data medium as claimed in the independent claims.

The method for identifying a characteristic profile of an R-wave in an EKG signal here comprises the following steps:
Recording a temporal sequence of measurement values,
Storing the measurement values with associated time value,
Identifying a number of values for identified temporal derivative and their respective time values in the stored measurement values,
Selecting one of the values for identified temporal derivative as an exemplary value, the selection of one of the values for identified temporal derivative being an exemplary value comprising at least one plausibility test,
Selecting a sub-sequence of the stored measurement values as the characteristic profile as a function of the time value associated with the exemplary value.

The combination of identifying possible values by way of their derivative with a plausibility test makes the method very robust. It can therefore be used reliably even with existing EKG signal interference to identify a characteristic profile of an R-wave of the EKG signal. For example if a patient is already present within the measurement volume of the magnetic resonance device, the electromagnetic fields of which cause T-wave overshoot in the EKG signal, it is still possible to identify a characteristic profile of an R-wave of the EKG signal reliably. This characteristic profile can then be used during an ongoing MR examination of the patient to compare current EKG signals with the previously determined characteristic profile of the R-wave. This allows particularly reliable detection of R-waves in the current EKG signal and therefore particularly reliable triggering of the MR examination, in particular as the comparison values obtained from the characteristic profile and the currently measured EKG signals are measured in the most similar conditions possible. If necessary the characteristic profile of the R-wave can also be determined again during the MR examination, without the patient having to be moved out of the magnetic resonance device. It is thus possible to respond to any changes occurring in the EKG measurement apparatus or the heartbeat (for example due to the patient sweating or feeling stressed) and associated changes in the EKG signal obtained. With a corresponding embodiment of the magnetic resonance device such a determination of the characteristic profile can conveniently be prompted from an operating console, for example by clicking on a corresponding software button.

In one advantageous exemplary embodiment the value for identified temporal derivative is the value for greatest temporal derivative. The value for greatest temporal derivative is very probably located on the rising edge of the R-wave (Q-R in FIG. 1) and therefore marks a region in the EKG signal, that is located shortly before the event of interest, the R-wave itself. If such a value is used to determine a characteristic profile of the EKG signal around the desired trigger time (R-wave), it is possible later to anticipate a possible R-wave in the current EKG signal by comparing a current signal with the characteristic profile. Other values for temporal derivative can also be used as identified values. For example extreme local values for temporal derivative or a smallest temporal derivative, which is very probably located on the falling edge of the R-wave in the S-valley. However the value for greatest temporal derivative is recommended due to its position before the trigger time to be detected and the fact that it is simple to calculate. Therefore values for greatest temporal derivative are generally referred to below rather than values for identified temporal derivative. If a value other than the identified value for temporal derivative is used, the following applies accordingly.

The identification of a number of values for greatest temporal derivative and their respective time values in the stored measurement values advantageously comprises a division of the storage unit containing the measurement values into storage sub-units. The storage sub-units here are advantageously selected so that they contain a number of measurement values that were recorded within a time period corresponding to a cardiac period. If just the value for greatest temporal derivative and its associated time value are now identified and stored in each storage sub-unit, it can be assumed with a high level of probability that this is a value on the ascent of the R-wave in the relevant cardiac period. The stored values for greatest temporal derivative therefore represent the edges of potential R-waves and thus potential R-waves.

In one advantageous embodiment of the method a sub-set to be assigned to an R-wave and its associated time values are selected according to a predefined rule from the number of values for greatest temporal derivative. A first preselection of possible values to be assigned with a high level of probability to an R-wave therefore takes place already. For example the identified values for greatest temporal derivative are compared with the maximum, in this instance greatest, value for the identified values for greatest temporal derivative and those that deviate as a maximum by a predefined percentage, e.g. maximum 65% or less, e.g. 35%, from the greatest value are selected. If a value for identified temporal derivative is sought other than the value for greatest temporal derivative, the maximum value is the one that satisfies the identified property to the greatest degree.

In an inventive plausibility test at least one temporal distance between two identified temporally successive values for greatest temporal derivative is advantageously identified and compared with at least one comparison value. The comparison value here is for example a mean value for a typical cardiac period.

Alternatively or additionally in a plausibility test temporal distances between two identified temporally successive values for greatest temporal derivative can be compared. It can thus be ensured that the time distance between two values identified as possible R-waves is similar, thereby satisfying the periodicity requirement.

An inventive computer program product can be loaded directly into a storage unit of a programmable processing unit of a magnetic resonance system and comprises program means for executing all the steps of the described method when the program is run in the processing unit of the magnetic resonance system.

An inventive electronically readable data medium comprises electronically readable control information stored thereon, which is embodied so that it performs the described method when the data medium is used in a processing unit of a magnetic resonance system.

The advantages and embodiments relating to the method apply similarly to the computer program product and the electronically readable data medium.

### BRIEF DESCRIPTION OF THE DRAWINGS

Further advantages and details of the present invention will emerge from the exemplary embodiments described below and with reference to the figures. The examples cited do not restrict the invention in any way. In the drawing:

FIG. 1 shows an example of an ideal profile of an EKG signal over time,

FIG. 2 shows a schematic diagram of an EKG measurement apparatus and a magnetic resonance device, in conjunction with which the method can be performed,

FIG. 3 shows a schematic diagram of a sequence of the inventive method.

### DETAILED DESCRIPTION OF THE INVENTION

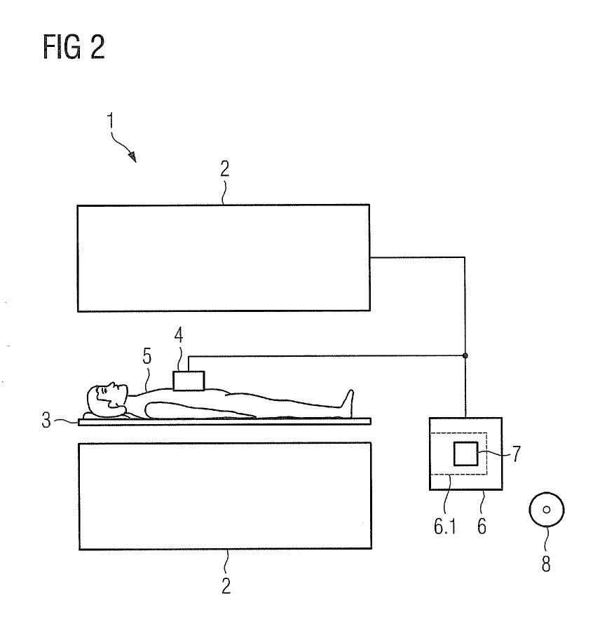

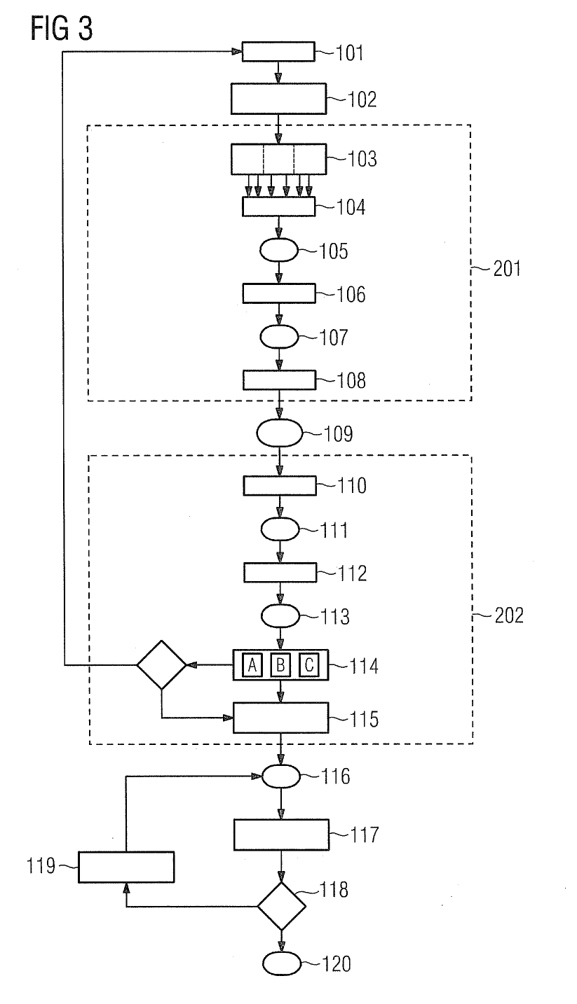

The inventive method is described below in conjunction with an EKG measurement apparatus and a magnetic resonance device 1 with reference to FIGS. 2 and 3.

As illustrated in FIG. 2, a patient 5 with an EKG measurement apparatus 4 positioned thereon is present in the magnetic resonance device 1 during an examination. The magnetic resonance device 1 is only illustrated schematically here by its magnet unit 2 and a patient couch 3, used to move the patient 5 into and out of the magnetic resonance device 1. The basis structure of a magnetic resonance device consisting of high-frequency coils and a gradient coil unit and the associated control units of a magnetic resonance device are known. The EKG measurement apparatus 4 is also only illustrated schematically as a block, since the basic structure of an EKG measurement apparatus with EKG electrodes and amplifier/filter units for measuring a voltage between two EKG electrodes is known.

According to the invention the EKG measurement apparatus 4 and the magnetic resonance device 1 are connected to a processing unit 6, which can receive data from the EKG measurement apparatus 4 and can communicate with the magnetic resonance device 1, for example a control unit of the same. The processing unit 6 here comprises at least one storage unit 6.1.

When an inventive computer program product 7 is loaded into the programmable processing unit 6 of the magnetic resonance device 1, the method described below can be performed when the program included on the computer program product 7 is run on the processing unit 6. Such a computer program product 7 can also be stored as electronically readable control information on an electronically readable data medium 8 and can permit performance of the method when the data medium 8 is used in the processing unit 6 of the magnetic resonance device 1.

FIG. 3 shows a schematic flow diagram of the inventive method, in which advantageous embodiments of the method are indicated.

A temporal sequence of measurement values, which represent an EKG signal measured using an EKG measurement apparatus for example as voltage values, is first recorded (block 101). The recorded measurement values are stored with an associated time value, which indicates the temporal relationship of the recorded measurement values to one another, in a storage unit (block 102). A sufficient number of measurement values should be recorded here. In particular the recorded temporal sequence of measurement values should advantageously include at least one cardiac period, but advantageously a number of cardiac periods, resulting in the required storage volume.

If we assume for example a minimum heart rate of 25 beats per minute (bpm), a cardiac period of 60 s/25=2.4 s (s, seconds) results. If for example at a sampling rate of 2.5 ms (ms, milliseconds), i.e. a recording of a measurement value every 2.5 ms, at least n (n=1, 2, 3, . . . ) cardiac periods are captured, this would produce a storage volume of n*2.4 s/2.5 ms=n*960 measurement values and the storage unit would cover a temporal sequence over a period of n*960*2.5 ms. It would generally be sufficient to record 2880 measurement values (n=3), corresponding to a sequence lasting 3*960*2.5 ms=7.2 s.

In block 201 the stored measurement values are used to identify a number of measurement values which, of the stored measurement values, have values for an identified, e.g. a greatest, temporal derivative (result 105) and therefore represent potential R-waves (see above). The respective derivative values are in turn stored with their associated time value, with, in a result 109, time values associated with stored values for greatest temporal derivative being stored for example in a temporally ascending order. In this process for example the time “0” can be assigned in the respectively last stored value.

In one advantageous embodiment the identification of the number of values for greatest temporal derivative and their respective time values in the stored measurement values 201 includes a division of a storage unit containing the measurement values into storage sub-units (block 103). It is advantageous here, depending on the sampling rate and an assumed maximum heart rate, for the storage unit to be divided into so many storage sub-units that the measurement values contained in a storage sub-unit cover maximum one full cardiac period or less.

In the aforementioned example of a storage unit containing n*960 measurement values, said storage unit can be divided for example into n*8 storage sub-units with 120 measurement values each. This ensures that even at a maximum assumed heart rate of 200 bpm, and therefore a cardiac period of 60 s/200=0.3 s (0.3 s/2.5 ms=120), measurement values for a maximum of the whole cardiac period or just one sub-period are contained in a storage sub-unit.

A number of values for greatest temporal derivative and their respective time values are now identified (block 104) and stored (result 105) in the measurement values stored in the storage unit. It is advantageous here for a value for greatest temporal derivative of the relevant storage sub-unit and its associated time value to be identified and stored in each storage sub-unit. This ensures that the identified values for greatest temporal derivative have a certain minimum temporal distance which, as in the case described above of storage sub-units covering maximum one cardiac period, is in the order of an expected temporal distance between two R-waves to be detected (temporal distance: one cardiac period). In the case of a (e.g. α=n*8) storage sub-units, the result 105 therefore contains α values for greatest temporal derivative and their associated time values. If the storage unit is not divided into storage sub-units, an adequate temporal distance between the values to be stored can be ensured in a different manner, for example by checking the time values, in some instances combined with a maximum number of values for greatest temporal derivative to be stored.

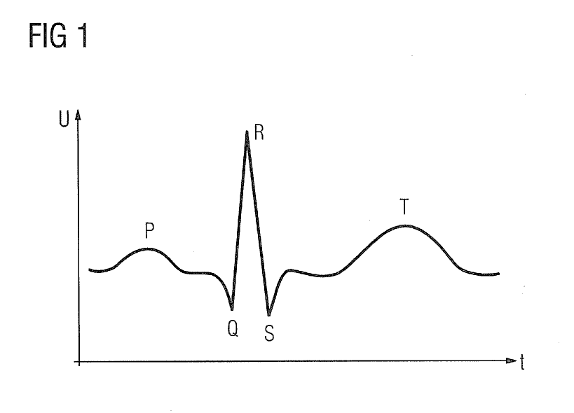

The steepest ascent in an EKG signal is normally located on the rising edge of the R-wave (see also FIG. 1), so it is anticipated that the stored values for greatest temporal derivative are also located on such a rising edge of an R-wave in the recorded EKG signal.

The descent of the R-wave into the S-valley also has a steep pitch, but with a different sign in front of it. In the present instance a derivative, described by a pitch, should have a positive sign so that values for greatest temporal derivative are always located on an ascent (e.g. Q-R edge, see FIG. 1) in the EKG signal. If it is not already possible to distinguish which derivative values represent an ascent and which a descent (signs unknown) and it is still necessary to ensure that only values on the aforementioned ascent and not on the descent into the S-valley are stored as values for greatest temporal derivative, it is in some instances possible to enquire about the dynamic of the measurement values temporally before the value identified first as the measurement value, at which the value identified as the value for greatest derivative is present, to decide whether the identified value is located on the rising edge of the R-wave or on the descent into the S-valley and to store it or reject it accordingly. If an identified value for temporal derivative is sought other than the value for greatest temporal derivative, similar enquiries can be undertaken.

In one advantageous embodiment the values for greatest temporal derivative obtained as result 105 are sorted and stored in a suitable, e.g. descending order, with the associated time values being sorted and stored in a similar manner (result 107), so that an assignment of derivative value to time value is still possible (block 106). Sorting can take place for example by allocating corresponding indices both to the derivative values and to the associated time values.

The sorting of the derivative values by size makes it possible to refine and reduce the number of these values to be interpreted as possibly belonging to an R-wave. Since, as mentioned above, real R-waves have the greatest ascent within an EKG signal, the greatest derivative values of the stored values for greatest temporal derivative should above all be considered as possible R-waves.

A sub-set is therefore advantageously selected from the number of values for greatest temporal derivative according to a predefined rule, the elements of which sub-set are to be assigned to an R-wave, and their associated time values (block 108).

The first N indices of the above derivative values sorted in descending order can therefore be identified in a simple manner as such a sub-set, the following applying: W_1*proc<W_i, where W_1 is the greatest of the stored derivative values. W_i is any stored derivative value and proc is a value between 0 and 1. The selection of the sub-set of values for greatest temporal derivative, which are to be assigned to an R-wave, therefore includes a comparison with the greatest value for temporal derivative W_1 of the identified values for greatest temporal derivative. The value W_1 is assumed to be located on an R-wave in every instance. The value proc therefore defines a barrier for by how many percent a further one of the stored derivative values potentially associated with an ascent of an R-wave can deviate from the maximum ascent W_1, to still be considered as possibly being associated with a further R-wave. For example proc=0.65 or proc=0.5 or a value between 0.65 and 0.35 can be set as proc. Values W_i<W_1*proc probably originate instead from other profiles in the EKG signal and are not considered further.

The values with W_i>W_1*proc as associated with the sub-set are stored with their associated time values t(W_i) (with W_i>W_1*proc) as result 109. Such a reduction of the values for greatest temporal derivative also reduces the further computation outlay.

A value can advantageously be selected from the values of the above sub-set (result 109) or from the originally identified values for greatest temporal derivative (result 105, see above) as an exemplary value, the selection of one of the values for greatest temporal derivative as the exemplary value including at least one plausibility test (block 202).

To this end, the time values stored in the result 109 can first be sorted into an order, e.g. ascending, (block 110) and stored as result 111. The selection of one of the values for greatest temporal derivative as an exemplary value therefore includes sorting the identified values for greatest temporal derivative e.g. in ascending order according to their time values.

If the storage unit has been divided into storage sub-units as described above and one value for greatest temporal derivative has been identified in each storage sub-unit, the result 109 comprises as many time values as there are storage sub-units a present (e.g. 8*n, see above). Also if a sub-set of N values of the stored values for greatest temporal derivative has also been selected, as described as further advantageous above, the result 109 comprises N time values.

When the storage unit is divided into storage sub-units it may be necessary to correct the result 109, specifically when a storage sub-unit limit is located on a rising edge of an R-wave in the recorded EKG signal, as it may then be possible that values associated with the same rising edge of an R-wave have been identified as values for greatest temporal derivative in both storage sub-units, as separated by the storage sub-unit limit.

This can advantageously be corrected in that an enquiry checks the respective temporal distances between the values of the result 111 for a predefined minimum length and, if the temporal sequence of two successive values in the result 111 is too close, optionally rejects the first or second of the two (block 112). If for example a minimum length of 40 ms is assumed for an R-wave, one of two successive values in the result 111, which are 40 ms or less apart from one another, is rejected. At a sampling rate of 2.5 ms this temporal distance corresponds to 16 sampling values. The accordingly corrected result 111 is stored as result 113 and now contains M inputs.

At least one plausibility test is now performed in block 114 as part of the selection of one of the values for greatest temporal derivative as an exemplary value.

A first possible plausibility test “A” could compare the number of inputs in the result 111 with a minimum number of R-waves anticipated in the recorded measurement values and in particular enquire whether M>=n, where M is the number of inputs in the result 111 and n, as set out above, is a minimum number of cardiac periods covered by the recorded measurement values. If the result 111 contains fewer inputs than there were cardiac periods covered by the recorded measurement values, not enough values of greatest ascending derivative were identified and the method starts again at block 101.

Further possible plausibility tests could include the identification of at least one temporal distance between two identified temporally successive values for greatest temporal derivative and a comparison of the identified distance with at least one comparison value. RR=Erg111_2−Erg111_1 is formed for example, where RR reflects the temporal distance between the values Erg111_2 and Erg111_1 stored at the second and at the first point in the result 111, and therefore the temporal distance from the first potential R-wave to the second. If further potential R-waves have been identified, any other distance between two successive potential R-waves can also be used.

The following might then have to apply for a plausibility test “B”: “upper barrier”>RR>“lower barrier”.

The identified time distance is therefore compared for example with an upper and lower comparison value. A cardiac period associated with an assumed minimum heart rate can in particular be used as the upper barrier. In the aforementioned example of a minimum heart rate of 25 bpm, which corresponds to a cardiac period of 2.4 s, this would be 2.4 s, which at a sampling rate of 2.5 ms corresponds to 960 measurement values. A cardiac period associated with an assumed maximum heart rate can similarly be used as the lower barrier. In the aforementioned example of a maximum heart rate of 200 bpm, this would be 0.3 s which at a sampling rate of 2.4 ms corresponds to 120 measurement values.

If at least three inputs are contained in the result 111 and therefore at least three potential R-waves are identified, for a further plausibility test “C” the identified temporal distance between two identified temporally successive values for greatest temporal derivative RR can also be compared with further temporal distances between two (other) identified temporally successive values for greatest temporal derivative RR_m=Erg111_m−Erg111_(m−1)(m=2, 3, . . . , M).

The purpose of the comparison in the plausibility test “C” here is for example to ensure adequate similarity of time differences between two successive potential R-waves, in accordance with the periodicity requirement predefined by the cardiac period. It may therefore be required that for all m the following applies RR_m<2,0*RR AND RR_m>0,40*RR.

Upper and lower limits as a function of a first temporal distance RR are thus created for the further temporal distances RR_m. In the above example the lower limit was selected so that the temporal distance RR_m between two successive potential R-waves is at least 40% of the first temporal distance RR. The upper limit was selected so that the temporal distance RR_m is not greater than twice the first temporal distance RR. Such an upper limit ensures that a temporal distance of perhaps two cardiac periods is still taken into account, should an actual R-wave between two values detected as potential R-waves not have been identified. The limits can be tailored to the relevant conditions and can be selected for example for the upper limit from values between 1.5*RR and 2.5*RR and for the lower limit from values between 0.35*RR and 0.5*RR.

If a plausibility test is not satisfied, the method continues with block 101 with the recording of new measurement values.

Once all the plausibility tests have been satisfied, a value is selected as an exemplary value from the M values representing potential R-waves contained in the result 111. One possible way of selecting an exemplary value is for example to select the value of the result 109 that is located at the point [M/2], where [*] is the Gaussian parenthesis and therefore optionally represents a rounding up or down. In other words the value for greatest temporal derivative that, among the values of the result 109 sorted in descending order by size, of the M values identified as a potential R-wave, has a mean dynamic is selected as the exemplary value. Thus the selection of one of the values for greatest temporal derivative as an exemplary value includes the identification of the value that has a mean dynamic of the values to be assigned to an R-wave (block 115). Such a selection of an exemplary value will therefore select the potential R-wave that has the closest possible relationship to all the potential R-waves contained in the result 111.

At least the time value associated with the value for greatest temporal derivative selected as the exemplary value is stored as result 116.

A sub-sequence of the measurement values stored in Block 102 is selected as the characteristic profile (block 117) as a function of the result 116, i.e. as a function of the time value associated with the exemplary value.

In one advantageous embodiment a sub-sequence of the stored measurement values selected in block 117 comprises a chronological sequence of stored measurement values, which include the measurement value to which the time value stored as result 116 corresponds. In other words the sub-sequence of stored measurement values selected as the characteristic profile comprises a chronological sequence of stored measurement values, which include the measurement value that has the same time value as the value selected as the exemplary value.

The sub-sequence selected as the characteristic profile therefore in each instance comprises at least a part of the rising R-edge, on which the value selected as the exemplary value is located.

The selection of a sub-sequence of stored measurement values as the characteristic profile also advantageously includes an identification of extreme values in the profile of the stored values before and after the exemplary value as the start and end values of the characteristic profile. This means that the rising R-edge, on which the value selected as the exemplary value is located, is selected as the characteristic profile, since the R-edge starts with a local minimum and ends with a local maximum. The characteristic profile can also optionally be selected as extending beyond the rising R-edge, e.g. by also including further measurement values in a predetermined time interval before the determined local minimum and/or after the determined local maximum and therefore before or after the rising R-edge in the characteristic profile. The respective selection of the characteristic profile around the measurement value determined as the exemplary value can be adjusted according to the desired processing of the characteristic profile, e.g. as a comparison curve for a later R-wave detection in subsequent EKG signal measurements. The characteristic profile is stored as result 120.

In one advantageous embodiment of the invention it is checked during identification of the characteristic profile by means of an enquiry 118 whether all the measurement values associated with the characteristic profile according to the criteria of block 117 are contained in the measurement values stored in block 102. If for example the value selected as the exemplary value is close to the start or end of the storage unit from block 102, not all the desired values might be present. In such an instance the result 116 can be modified so that instead of the time value selected in block 115, which is located at point [M/2] of the result 109, the time value located at point [M/2]+1 or, e.g. in a second pass through the block 119, at point [M/2]−1 of the result 109 is selected, before the characteristic profile is again determined in block 117. In this manner a presumably similarly suitable value is selected as the exemplary value, which with a high level of probability is also not located at one of the edges of the storage unit from block 102 and therefore allows the full desired characteristic profile to be determined.

With the proposed method the storage unit in block 102 can advantageously be continuously updated; in other words new measurement values are continuously stored, the number of measurement values stored in the storage unit being kept constant, in that the oldest stored measurement value in each instance is rejected as soon as a new (latest) measurement value is added. The method can be started again when a sufficient number of new measurement values has been stored to fill a storage sub-unit. This means that the method always analyzes current measurement values.

1.-15. (canceled)

16. A method for identifying a characteristic profile of an R-wave in an EKG signal, comprising:
recording a temporal sequence of measurement values;
storing the measurement values with associated time values;
identifying a number of values and respective time values from the stored measurement values;
selecting a value for an identified temporal derivative from the identified values as an exemplary value by a plausibility test; and
selecting a sub-sequence from the stored measurement values as the characteristic profile as a function of a time value associated with the exemplary value.

17. The method as claimed in claim 16, wherein the value for the identified temporal derivative is a value for greatest temporal derivative.

18. The method as claimed in claim 16, wherein the identified values and the respective time values are identified by dividing a storage unit containing the measurement values into storage sub-units.

19. The method as claimed in claim 18, wherein the value for the identified temporal derivative and the time value associated with the exemplary value are identified and stored in each of the storage sub-units.

20. The method as claimed in claim 16, wherein the identified values are stored in an order according to the associated time values.

21. The method as claimed in claim 16, wherein the sub-sequence is selected according to a predefined rule from the identified values for greatest temporal derivative.

22. The method as claimed in claim 21, wherein the sub-sequence is selected by comparing the identified values with a maximum value of the identified values.

23. The method as claimed in claim 16, wherein the value for the identified temporal derivative is selected by sorting the identified values according to the associated time values.

24. The method as claimed in claim 16, wherein a temporal distance between two temporally successive values of the identified values is identified and compared with a comparison value during the plausibility test.

25. The method as claimed in claim 24, wherein the comparison value is a mean dynamic value of the identified values

26. The method as claimed in claim 16, wherein temporal distances between two temporally successive values of the identified values are identified and compared with each other during the plausibility test.

27. The method as claimed in claim 16, wherein the sub-sequence comprises a chronological sequence of the stored measurement values having a same time value with the exemplary value.

28. The method as claimed in claim 16, wherein a minimum value and a maximum value in the stored measurement values on which the exemplary value is located are determined.

29. The method as claimed in claim 28, wherein the sub-sequence stars at the minimum value and ends at the maximum value.

30. A computer program product loaded in a processing unit of a magnetic resonance system for identifying a characteristic profile of an R-wave in an EKG signal, the computer program product when executed in the processing unit executing steps comprising:
recording a temporal sequence of measurement values;
storing the measurement values with associated time values;
identifying a number of values and respective time values from the stored measurement values;
selecting a value for an identified temporal derivative from the identified values as an exemplary value by a plausibility test; and
selecting a sub-sequence from the stored measurement values as the characteristic profile as a function of a time value associated with the exemplary value.

31. An electronically readable data medium for identifying a characteristic profile of an R-wave in an EKG signal, the electronically readable data medium comprising a computer program product when executed in a processing unit of a magnetic resonance system executing steps comprising:
recording a temporal sequence of measurement values;
storing the measurement values with associated time values;
identifying a number of values and respective time values from the stored measurement values;
selecting a value for an identified temporal derivative from the identified values as an exemplary value by a plausibility test; and
selecting a sub-sequence from the stored measurement values as the characteristic profile as a function of a time value associated with the exemplary value.
